(19) United States
(12) Patent Application Publication
(10) Pub. No.: US 2010/0104603 A1
(43) Pub. Date: Apr. 29, 2010

(54) USE OF STAPHYLOCOCCAL SUPERANTIGEN-LIKE PROTEIN 5 (SSL5) IN MEDICINE

(76) Inventors: Carla J.C. De Haas, Bunnik (NL); Jovanka Bestebroer, Utrecht (NL); Cornelis Petrus Maria Van Kessel, Bunnik (NL); Johannes Antonius Gerardus Van Strijp, Odijk (NL)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/296,655

(22) PCT Filed: Apr. 13, 2007
(86) PCT No.: PCT/EP07/03290

§ 371 Date: Sep. 23, 2009

(30) Foreign Application Priority Data
Apr. 13, 2006 (EP) 06007815.1

(51) Int. Cl. A61K 39/085; C07K 14/31; A61P 37/06

(52) U.S. Cl. 424/243.1; 530/350

(57) ABSTRACT

The present invention relates to Staphylococcal superantigen-like protein 5 (SSL5) or homologues or derivatives thereof for use in medicine, in particular for use in the treatment of indications involving an excessive recruitment of leukocytes, such as stroke, perfusion/ischemia, transplant rejection, rheumatoid arthritis. The invention further relates to a pharmaceutical composition comprising SSL5 and a suitable excipient. The invention also provides the use of SSL5 for the preparation of a medicament for treatment of indications involving an excessive recruitment of leukocytes to a site of tissue damage, such as stroke, reperfusion/ischemic, transplant rejection and rheumatoid arthritis.

The present invention relates to the new use of the staphylococcal superantigen-like protein 5 (SSL5) in medicine, in particular for the treatment of indications involving an excessive recruitment of leukocytes to a site of tissue damage, such as stroke, reperfusion/ischemia, transplant rejection and rheumatoid arthritis.

Staphylococcus aureus is a common human pathogen that induces both community-acquired and nosocomial infections. This Gram-positive bacterium is well known for its suppurative diseases as skin-limited abscesses and boils, and more seriously endocarditis, sepsis and toxic shock syndrome. Its invasiveness is ascribed to the production of a wide repertoire of virulence factors that interfere with the host defense. For that reason, the bacterium produces cell-surface expressed as well as secreted proteins. Important cell surface-associated proteins include Protein A, which has specificity to the Fc region of immunoglobulins of different classes and thereby capture the immunoglobulins from phagocytic cells. Superantigens (SAg) constitute a large portion of the secreted arsenal of staphylococci in modulation of immune responses. They trigger non-specific activation of T lymphocytes by binding to the T cell receptor (TCR) and major histocompatibility complex (MHC) class II on antigen presenting cells (APC) outside the antigen-binding cleft.

Chemotaxis Inhibitory Protein of S. aureus (CHIPS), another excreted virulence factor of S. aureus was recently described. CHIPS is known to inhibit fMLP- and C5a-induced responses in neutrophils through binding directly to the formyl peptide receptor (FPR) and C5a receptor (C5aR), respectively. Thereby, CHIPS hampers the initial activation and migration of neutrophils to the site of infection, and thus it protects clearance of S. aureus by innate immune cells. Recently, the structure of CHIPS consisting of residues 31 to 121 (CHIPS₃₁₋₁₂₁) was resolved. This protein is composed of an α-helix packed onto a four-stranded antiparallel β-sheet, a domain also encountered in the C-terminal domain of SAgs. This protein also revealed to be homologous to the C-terminal domain of Staphylococcal superantigen-like (SSL) 5 and SSL7.

SSLs are a family of secreted proteins identified through sequence homology to staphylococcal and streptococcal superantigens. Eleven different SSLs exist that are encoded on Staphylococcal pathogenicity island 2 (SaPI2) in a conserved order. Staphylococci contain seven to eleven different SSLs, and their homology varies between 36 to 67%. Allelic variants, however, show 85 to 100% homology. Determination of the crystal structures of SSL5 and SSL7 also revealed their high structural homology to SAgs; the N-terminal oligosaccharide binding (OB)-fold and the C-terminal β-grasp domain characteristic for SAgs are also observed for SSLs. However, residues important for MHC class II and TCR binding of SAgs are not conserved in SSLs, which may explain their inability to induce superantigenic activities.

Recently, binding of complement factor 5 (C5) and IgA by SSL7 was described, suggesting a role for SSLs in staphylococcal defense against host immune responses. SSL7 was subsequently found to bind the Cα2/Cα3 interface of IgA Fc, which is the adhesion site of the FcαRI. So far, no other functions have been linked for the SSLs.

Neutrophil recruitment to sites of infection is a multistep process. The initial tethering and rolling of neutrophils on the endothelium of vessel walls during inflammation are mediated by P-selectin, a member of the selectin family that also includes E-selectin and L-selectin. P-selectin is stored in α-granules of platelets and Weibel-Palade bodies of endothelial cells, and is rapidly translocated to the cell surface after stimulation with thrombin and histamine.

PSGL-1 (P-selectin glycoprotein ligand-1) has been identified as the principle ligand for P-selectin. PSGL-1 is a disulfide-linked homodimer consisting of two glycoprotein chains with a molecular mass of 120 kD each. It is heavily glycosylated and contains sialylated fucosylated O-linked glycans, which terminate in the sialyl Lewis x (sLex).

Further post-translational modifications include up to three N-linked glycans and sulfation of at least one of the three tyrosines at the distal end of PSGL-1. PSGL-1 is expressed on most leukocytes, including neutrophils, monocytes, and T lymphocytes, and has been shown to mediate rolling of neutrophils on P-selectin in vitro and in vivo.

Chemokines are a family of small cytokines secreted by cells. They are classified as chemokines according to shared structural characteristics such as small size (they are all approximately 8-10 kilodaltons) and cysteine residues in conserved locations that determine the characteristic 3-dimensional shape.

Chemokines are categorized into four groups depending on the spacing of the first two cysteine residues. The CC chemokines (or β-chemokines) have two adjacent cysteines near their amino terminus (for example RANTES). The two N-terminal cysteines of CXC chemokines (or α-chemokines) are separated by one amino acid (for example IL8). The third group of chemokines is known as the C chemokines (or γ chemokines), and is unlike all other chemokines in that it has only two cysteines (for example Lymphotactin). A fourth group has three amino acids between the two cysteines and is termed CX3C chemokine (or δ-chemokines). The only CX3C chemokine discovered to date is called fractalkine.

Chemokine receptors are G-protein-coupled 7-transmembrane receptors expressed on the surfaces of certain cells. They are triggered by chemokines and as a consequence trigger a flux in intracellular calcium (Ca²⁺) ions (calcium signalling), which generates a chemotactic response of that cell, thus trafficking the cell to a desired location within the tissue. These chemokine receptors are divided into different families according to which family of chemokines they bind (CCR, CXCR, CR, or CX3CR).

Glycosaminoglycans or GAGs cover the surface of endothelial cells. They bind excreted chemokines and present these chemokines to rolling phagocytes. In doing so, they can activate rolling neutrophils and cause activation of Beta-2 integrins on the phagocyte surface. In turn, the phagocytes can now firmly adhere to the endothelial cells (e.g. by Beta-2 integrin to ICAM-1 interactions). This is the next and crucial step in the transmigration process, that in the end will lead to the translocation of blood phagocytes to the site of infection or, in inflammatory diseases, to the site of inflammation.

Several chronic and acute diseases, such as stroke, reperfusion/ischemia, transplant rejection, and rheumatoid arthritis are caused by an excessive recruitment of leukocytes to the site of tissue damage. Defining an inhibitor for the interaction between PSGL-1 and P-selectin is therefore an attractive therapeutic approach. It is even more desirable to define an inhibitor that blocks both the PSGL-1 to P-selectin interaction and the subsequent chemokine activation of phagocytes by chemokines.

In the research that led to the present invention it was found that SSL5 specifically binds PSGL-1 and functionally inhibits PSGL-1-mediated adhesion of neutrophils to P-selectin. It was furthermore found that PSGL-1 bound SSL5 binds chemokines thus inhibiting another step in the transmigration process.

The mechanism of action of SSL5 in its anti-inflammatory action and thus the role for SSL5 in the inhibition of phagocyte extravasation is as follows. Initial neutrophil rolling on activated endothelial cells at inflamed sites is mediated by the interaction of PSGL-1 and P-selectin. Rolling allows for encounter of activating signals as IL-8 on the endothelial lining (bound to GAG's), which induce cell activation. Subsequent integrin upregulation enables firm adhesion of the cells and allows for their transmigration through the endothelial lining.

SSL5 inhibits the two initial steps important in cell extravasation. Firstly, it inhibits neutrophil rolling by binding to PSGL-1. Secondly, SSL5 inhibits cell activation by binding to PSGL-1 and capturing the chemokines away from the chemokine receptors.

Thus, SSL5 inhibits the first two crucial steps in phagocyte extravasation. It inhibits the interaction of P-selectin with PSGL-1 and any other heavily and properly glycosylated phagocyte surface molecule. In doing so it also gains affinity for GAG-bound chemokines of at least the CC, CXC and the CX3C class and displaces these from the GAGs, keeping them bound to the PSGL-1-SSL5 complex. Now also the second step in cell migration and cell activation is abrogated. The combination of these two effects make SSL5 a very strong, highly potent, and completely unique anti-inflammatory molecule.

The invention thus relates to SSL5 or homologues or derivates thereof for use in medicine and more in particular for use in the treatment of indications involving excessive leukocyte recruitment, in particular stroke, reperfusion/ischemia, transplant rejection and rheumatoid arthritis.

The invention further relates to the use of SSL5 as an anti-inflammatory compound.

According to a first specific embodiment the invention relates to the use of SSL5 as an inhibitor of P-selectin glycoprotein ligand-1 (PSGL-1).

According to another specific embodiment the invention relates to the use of SSL5 as an inhibitor of chemokine stimulation of phagocytes.

In a further embodiment the invention relates to the use of SSL5 for inhibiting the interaction of P-selectin with PSGL-1 and by capturing GAG-bound chemokines.

SSL5 is known and described in Arcus et al., J. Biol. Chem. (2002) 277(35):32274-81 identified therein under the name SETS. The International Nomenclature Committee for Staphylococcal Superantigen Nomenclature has recommended that the SETs should be renamed staphylococcal superantigen-like proteins (SSLs) and that the genes should be designated SSL1 to SSL11 in clockwise order from the replication origin of the chromosome based on homology to the full complement of genes found in strain MW2. The amino acid sequence of SSL5 from S. aureus strain NTCT 8325 is:

(SEQ ID NO: 1)

SEHKAKYENVTKDIFDLRDYYSGASKELKNVTGYRYSKGGKHYLIFDKNR
KFTRVQIFGKDIERFKARKNPGLDIFVVKEAENRNGTVFSYGGVTKKNQD
AYYDYINAPRFQIKRDEGDGIATYGRVHYIYKEEISLKELDFKLRQYLIQ
NFDLYKKFPKDSKIKVIMKDGGYYTFELNKKLQTNRMSDVIDGRNIEKIE
ANIR

According to the invention also homologues of SSL5 and derivatives thereof can be used. Such homologues or derivatives must be functional. Derivatives may for example be fragments, such as peptides, truncated proteins, chimeric proteins comprising at least a functional part of SSL5 and another part, or peptidomimetic versions of the protein.

More specifically derivatives comprise polypeptides or peptides that comprise fewer amino acids than the full length SSL5 but still inhibit the interaction of PSGL-1 on phagocytes with P-selectin and still bind chemokines. Such derivatives preferably comprise a stretch of consecutive amino acids but combinations of active domains, optionally spaced by linkers, are also possible. The skilled person is very well capable of defining such derivatives on the basis of the SSL5 sequence of SEQ ID NO:1 and testing the thus defined derivative for the required activity as described in the Examples.

In some cases the potential for use of (poly)peptides in drugs may be limited for several reasons. In particular peptides may for example be too hydrophilic to pass membranes like the cell-membrane and the blood-brain barrier, and may be rapidly excreted from the body by the kidneys and the liver, resulting in a low bioavailability. Furthermore, they may suffer from a poor biostability and chemical stability since they may be quickly degraded by proteases, e.g. in the gastro-intestinal tract. Also, peptides generally are flexible compounds which can assume thousands of conformations. The bioactive conformation usually is only one of these possibilities, which sometimes might lead to a poor selectivity and affinity for the target receptor. Finally, the potency of the peptides may not be sufficient for therapeutical purposes.

As a result of the above described drawbacks, (poly)peptides are sometimes mainly used as sources for designing other drugs, and not as actual drugs themselves. In such case it is desirable to develop compounds in which these drawbacks have been reduced. Alternatives for peptides are the so-called peptidomimetics. Peptidomimetics based on SSL5 are also part of this application. In that case, one or more of the amino acids in SSL5 or a derivative thereof are substituted with peptidomimetic building blocks.

In general, peptidomimetics can be classified into two categories. The first consists of compounds with non-peptidelike structures, often scaffolds onto which pharmacophoric groups have been attached. Thus, they are low molecular-weight compounds and bear no structural resemblance to the native peptides, resulting in an increased stability towards proteolytic enzymes.

The second main class of peptidomimetics consists of compounds of a modular construction comparable to that of peptides, i.e. oligomeric peptidomimetics. These compounds can be obtained by modification of either the peptide side chains or the peptide backbone. Peptidomimetics of the latter category can be considered to be derived of peptides by replacement of the amide bond with other moieties. As a result, the compounds are expected to be less sensitive to degradation by proteases. Modification of the amide bond also influences other characteristics such as lipophilicity, hydrogen bonding capacity and conformational flexibility, which in favourable cases may result in an overall improved pharmacological and/or pharmaceutical profile of the compound.

Oligomeric peptidomimetics can in principle be prepared starting from monomeric building blocks in repeating cycles of reaction steps. Therefore, these compounds may be suitable for automated synthesis analogous to the well-established preparation of peptides in peptide synthesizers. Another application of the monomeric building blocks lies in the preparation of peptide/peptidomimetic hybrids, combining natural amino acids and peptidomimetic building blocks to give products in which only some of the amide bonds have been replaced. This may result in compounds which differ sufficiently from the native peptide to obtain an increased biostability, but still possess enough resemblance to the original structure to retain the biological activity.

Suitable peptidomimetic building blocks for use in the invention are amide bond surrogates, such as the oligo-8-peptides (Juaristi, E. Enantioselective Synthesis of b-Amino Acids; Wiley-VCH: New York, 1996), vinylogous peptides (Hagihari, M. et al., J. Am. Chem. Soc. 1992, 114, 10672-10674), peptoids (Simon, R. J. et al., Proc. Natl. Acad. Sci. USA 1992, 89, 9367-9371; Zuckermann, R. N. et al., J. Med. Chem. 1994, 37, 2678-2685; Kruijtzer, J. A. W. & Liskamp, R. M. J. Tetrahedron Lett. 1995, 36, 6969-6972); Kruijtzer, J. A. W. Thesis; Utrecht University, 1996; Kruijtzer, J. A. W. et al., Chem. Eur. J. 1998, 4, 1570-1580), oligosulfones (Sommerfield, T. & Seebach, D. Angew. Chem., Int. Ed. Eng. 1995, 34, 553-554), phosphodiesters (Lin, P. S.; Ganesan, A. Bioorg. Med. Chem. Lett. 1998, 8, 511-514), oligosulfonamides (Moree, W. J. et al., Tetrahedron Lett. 1991, 32, 409-412; Moree, W. J. et al., Tetrahedron Lett. 1992, 33, 6389-6392; Moree, W. J. et al., Tetrahedron 1993, 49, 1133-1150; Moree, W. J. Thesis; Leiden University, 1994; Moree, W. J. et al., J. Org. Chem. 1995, 60, 5157-5169; de Bont, D. B. A. et al., Bioorg. Med. Chem. Lett. 1996, 6, 3035-3040; de Bont, D. B. A. et al., Bioorg. Med. Chem. 1996, 4, 667-672; Löwik, D. W. P. M. Thesis; Utrecht University, 1998), peptoid sulfonamides (van Ameijde, J. & Liskamp, R. M. J. Tetrahedron Lett. 2000, 41, 1103-1106), vinylogous sulfonamides (Gennari, C. et al., Eur. J. Org. Chem. 1998, 2437-2449), azatides (or hydrazinopeptides) (Han, H. & Janda, K. D. J. Am. Chem. Soc. 1996, 118, 2539-2544), oligocarbamates (Paikoff, S. J. et al., Tetrahedron Lett. 1996, 37, 5653-5656; Cho, C. Y. et al., Science 1993, 261, 1303-1305), ureapeptoids (Kruijtzer, J. A. W. et al., Tetrahedron Lett. 1997, 38, 5335-5338; Wilson, M. E. & Nowick, J. S. Tetrahedron Lett. 1998, 39, 6613-6616) and oligopyrrolinones (Smith III, A. B. et al., J. Am. Chem. Soc. 1992, 114, 10672-10674).

The vinylogous peptides and oligopyrrolinones have been developed in order to be able to form secondary structures (β-strand conformations) similar to those of peptides, or mimic secondary structures of peptides. All these oligomeric peptidomimetics are expected to be resistant to proteases and can be assembled in high-yielding coupling reactions from optically active monomers (except the peptoids).

Peptidosulfonamides are composed of α- or β-substituted amino ethane sulfonamides containing one or more sulfonamide transition-state isosteres, as an analog of the hydrolysis of the amide bond. Peptide analogs containing a transition-state analog of the hydrolysis of the amide bond have found a widespread use in the development of protease inhibitor.

Another approach to develop oligomeric peptidomimetics is to completely modify the peptide backbone by replacement of all amide bonds by nonhydrolyzable surrogates e.g. carbamate, sulfone, urea and sulfonamide groups. Such oligomeric peptidomimetics may have an increased metabolic stability. Recently, an amide-based alternative oligomeric peptidomimetics has been designed viz. N-substituted Glycine-oligopeptides, the so-called peptoids. Peptoids are characterized by the presence of the amino acid side chain on the amide nitrogen as opposed to being present on the α-C-atom in a peptide, which leads to an increased metabolic stability, as well as removal of the backbone chirality. The absence of the chiral α-C atom can be considered as an advantage because spatial restrictions which are present in peptides do not exist when dealing with peptoids. Furthermore, the space between the side chain and the carbonyl group in a peptoid is identical to that in a peptide. Despite the differences between peptides and peptoids, they have been shown to give rise to biologically active compounds.

Translation of a peptide chain into a peptoid peptidomimetic may result in either a peptoid (direct-translation) or a retropeptoid (retro-sequence). In the latter category the relative orientation of the carbonyl groups to the side chains is maintained leading to a better resemblance to the parent peptide.

Review articles about peptidomimetics that are incorporated herein by reference are:

Adang, A. E. P. et al.; Recl. Tray. Chim. Pays-Bas 1994, 113, 63-78; Giannis, A. & Kolter, T. Angew. Chem. Int. Ed. Engl. 1993, 32, 1244-1267; Moos, W. H. et al., Annu. Rep. Med. Chem. 1993, 28, 315-324; Gallop, M. A. et al., J. Med. Chem. 1994, 37, 1233-1251; Olson, G. L. et al., J. Med. Chem. 1993, 36, 3039-30304; Liskamp, R. M. J. Recl. Tray. Chim. Pays-Bas 1994, 113, 1-19; Liskamp, R. M. J. Angew. Chem. Int. Ed. Engl. 1994, 33, 305-307; Gante, J. Angew. Chem. Int. Ed. Engl. 1994, 33, 1699-1720; Gordon, E. M. et al., Med. Chem. 1994, 37, 1385-1401; and Liskamp, R. M. J. Angew. Chem. Int. Ed. Engl. 1994, 33, 633-636.

The invention thus furthermore relates to molecules that are not (poly)peptides themselves but have a structure and function similar to those of SSL5 or derivatives thereof.

Homologues are intended to encompass allelic variants of the S. aureus SSL5 as well as homologues from other bacteria strains. An example of a homologue is:

(SEQ ID NO: 2)

SEHKAKYENV TKDIFDLRDY YSGASKELKN VTGYRYSKGG
KHYLIFDKHQ KFTRIQIFGK DIERLKTRKN PGLDIFVVKE
AENRNGTVFS YGGVTKKNQG AYYDYLNAPK FVIKKEVDAG
VYTHVKRHYI YKEEVSLKEL DFKLRQYLIQ NFDLYKKFPK
DSKIKVIMKD GGYYTFELNK KLQPHRMSDV IDGRNIEKME ANIR

The invention further relates to pharmaceutical compositions comprising a suitable excipient and a therapeutically active amount of SSL5 or a homologue or derivative thereof. The skilled person in the field of pharmacy will be able to define suitable excipients and dosage forms as well as administration regimes for the treatment of indications involving excessive leukocyte recruitment, such as stroke, reperfusion/ischemia, transplant rejection and rheumatoid arthritis.

According to a further aspect thereof the invention relates to the use of SSL5 or a homologue or derivative thereof for the preparation of a medicament for the treatment of indications involving an excessive recruitment of leukocytes, such as stroke, reperfusion/ischemia, transplant rejection and rheumatoid arthritis.

Chemokine-chemokine receptor interactions as well as the intervention of P-selectin to PSGL-1 interactions have both been described in literature as highly important strategies for intervention of a variety of inflammatory disorders as well as cancer. Both have been shown in various animal models and in knock out studies to be strategic points to intervene with inflammation. The aim is now to develop good inhibitors to achieve this intervention. Here we describe a molecule that combines these functions, a broad chemokine inhibitor in combination with a P-selectin-PSGL-1 inhibitor. (Giles R, Loberg R D. Can we target the chemokine network for cancer therapeutics? Curr Cancer Drug Targets. 6(8):659-70 (2006); Rychly J, Nebe B. Therapeutic strategies in autoimmune diseases by interfering with leukocyte endothelium interaction. Curr Pharm Des. 12(29):3799-806 (2006); Walsh G M. Targeting airway inflammation: novel therapies for the treatment of asthma. Curr Med. Chem. 13(25):3105-11 (2006); Kobayashi Y. Neutrophil infiltration and chemokines. Crit. Rev Immunol. 26(4):307-16 (2006); Tremoulet A H, Albani S, Novel therapies for rheumatoid arthritis. Expert Opin Investig Drugs. 15(11):1427-41 (2006); Zoliner T M, Asadullah K, Schon M P. Targeting leukocyte trafficking to inflamed skin: still an attractive therapeutic approach? Exp Dermatol. 16(1):1-12 (2007); Rychly J, Nebe B. Therapeutic strategies in autoimmune diseases by interfering with leukocyte endothelium interaction. Curr Pharm Des. 12(29):3799-806 (2006); Kaneider N C, Leger A J, Kuliopulos A. Therapeutic targeting of molecules involved in leukocyte-endothelial cell interactions. FEBS J. 273(19):4416-24 (2006); Romano S J. Selectin antagonists: therapeutic potential in asthma and COPD. Treat Respir Med. 4(2):85-94 (2005).

The invention will be further illustrated in the Examples that follow and that are given for illustration purposes only and are not intended to limit the invention in any way. Reference is made to the following figures.

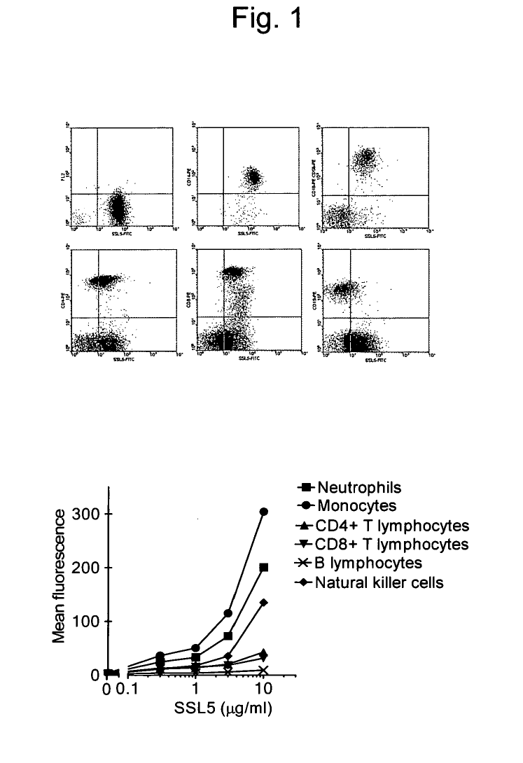

FIG. 1. Binding of SSL5 to leukocytes. Two-color flow cytometry was used to analyse SSL5 binding to different leukocyte subpopulations. Leukocytes were incubated with a concentration range of FITC-conjugated SSL5 (0.3-10 μg/ml) for 30 min at 4° C. To differentiate for specific leukocyte subpopulations, monocytes, T lymphocytes, and B lymphocytes were concurrently stained with anti-CD14-PE, anti-CD4-PE or anti-CD8-PE, and anti-CD19-PE (i.e. PE-conjugated antibodies directed against CD14, CD4 or CD8, and CD19) respectively. Natural killer cells were first negatively selected for binding of anti-CD3-Cy and then positively selected for binding anti-CD16-PE and anti-CD56-PE. Neutrophils were selected by gating. The data are representative of three independent experiments.

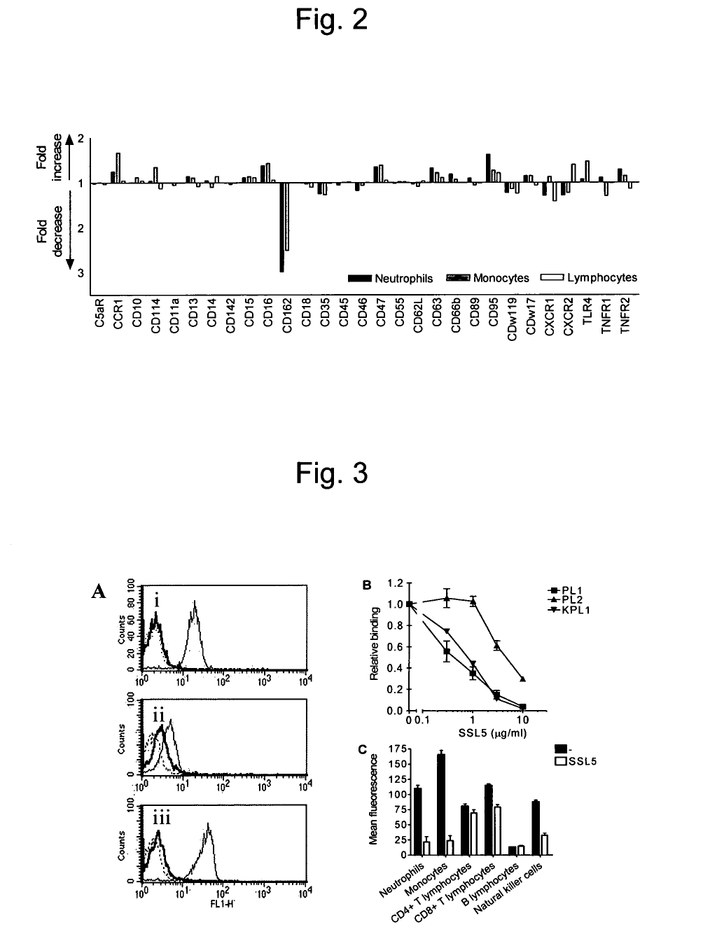

FIG. 2. Competition for receptor binding. To determine a putative receptor for SSL5, a mixture of neutrophils (5×10⁶ cells/ml) and PBMC (1×10⁷ cells/ml) was incubated with 10 μg/ml SSL5 for 15 minutes on ice in the presence of 10% heat-inactivated human pooled serum.

Subsequently, FITC-, PE- and APC-conjugated mAbs directed against a series of cell surface receptors were added for 30 minutes on ice. After washing, fluorescence was measured using flow cytometry. Neutrophils, monocytes and lymphocytes were selected by gating.

FIG. 3. Competition of SSL5 in binding of anti-PSGL-1 mAbs to neutrophils. (A) Neutrophils were treated without (thin continuous line) or with 10 μg/ml SSL5 (thick continuous line) for 30 min at 4° C. After washing, the cells were treated with 1 μg/ml anti-PSGL-1 PL1(i), PL2(ii), or KPL1(iii). Bound antibodies were detected with FITC-conjugated goat anti-mouse IgG. Dashed line represents control-treated cells. (B) Binding of 1 ug/ml KPL1, PL1 or PL2 to neutrophils pretreated with a concentration range of SSL5 (0.3-10 μg/ml). Data represent relative binding of mAbs compared to control-treated cells and are mean values ±SEM of three independent experiments. (C) Two-color flowcytometry was used to analyze binding of 1 μg/ml KPL1 to different leukocyte subpopulations in presence (white bars) or absence (black bars) of 10 μg/ml SSL5. Neutrophils were selected on gating, while monocytes, T lymphocytes, and B lymphocytes were stained with anti-CD14-PE, anti-CD4-PE or anti-CD8-PE, and anti-CD19-PE, respectively. Natural killer cells were first negatively selected for anti-CD3-Cy and then positively selected for anti-CD16-PE and anti-CD56-PE. The data represent mean fluorescence of detected KPL1 and are mean values ±SEM of three independent experiments.

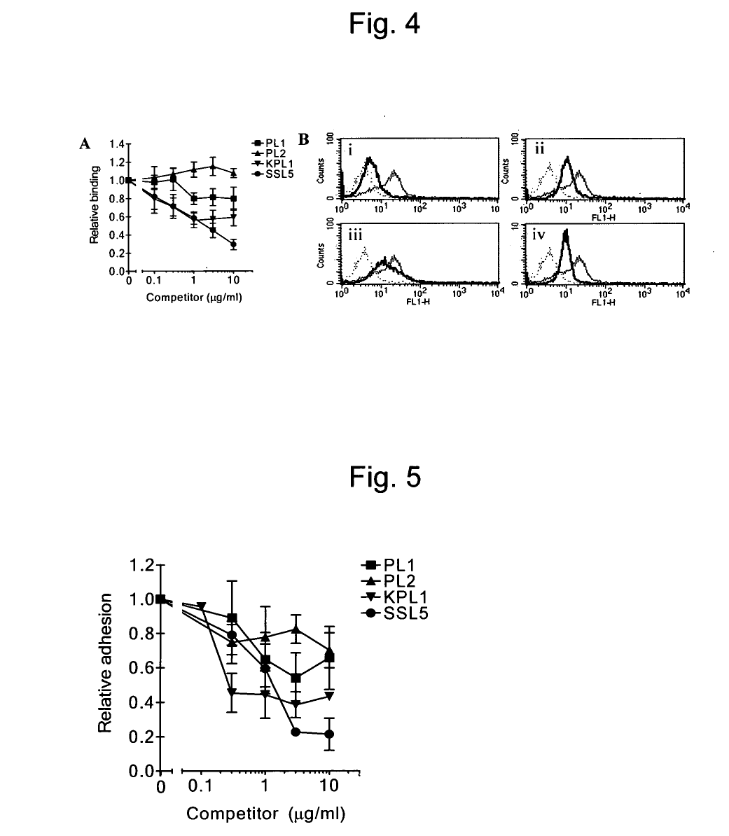

FIG. 4. Competition of SSL5 in binding of P-selectin-Fc chimera (PselFc) to neutrophil. (A) Neutrophils were incubated with 0.3-10 μg/ml SSL5, or PSGL-1 mAbs PL1, PL2 or KPL1 for 30 min at 4° C. After washing, the cells were treated with 1 μg/ml PselFc. Bound PselFc was detected with FITC-conjugated goat anti-human IgG. The data represent relative binding of PselFc compared to control-treated cells and are mean values ±SEM of three independent experiments. (B) Histograms depict binding of 1 μg/ml PselFc to neutrophils in absence (thin continuous line) and presence of 10 μg/ml SSL5 (i), PL1 (ii), PL2 (iii) and KPL1 (iv) (thick continuous line). Dashed line represents control-treated cells.

FIG. 5. Effect of SSL5 on adhesion of neutrophils under static conditions. Calcein-labeled neutrophils were incubated with 0.3-10 μg/ml SSL5 or PSGL-1 mAbs PL1, PL2, or KPL1 for 10 min. Subsequently, the neutrophils were incubated in duplicate wells for 15 min in a 96-wells microtiterplate to which PselFc was immobilized. After washing, bound neutrophils were quantified using a microplate reader. The data represent relative adhesion of neutrophils compared to control-treated cells and are mean values ±SEM of three independent experiments.

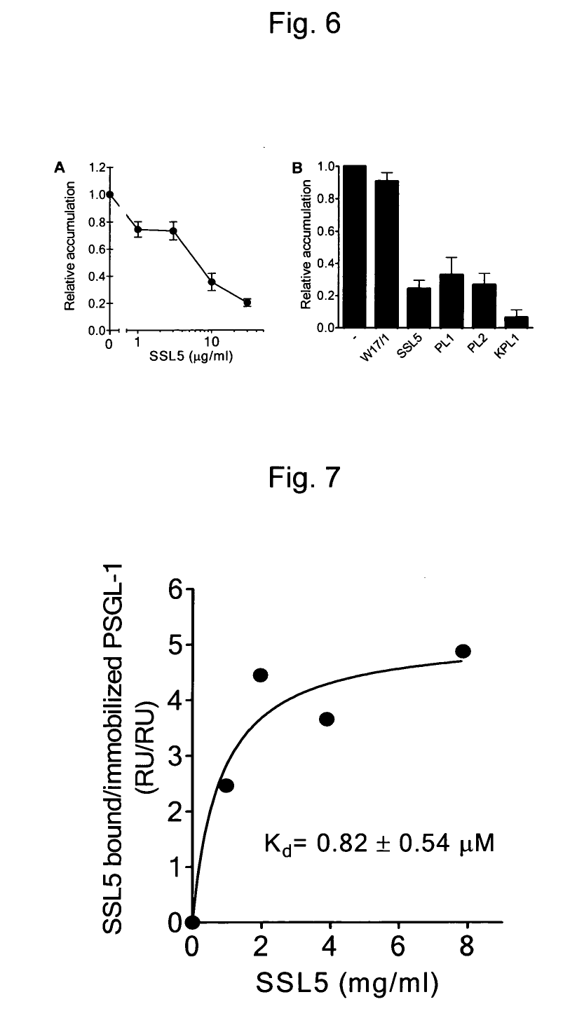

FIG. 6. Effect of SSL5 on rolling of neutrophils under shear conditions. Neutrophils were treated with SSL5 or PSGL-1 mAbs for 15 min at 37° C. Subsequently, the neutrophils were perfused over glass cover slips coated with 10 μg/ml PselFc at a shear stress of 200/s during 5 min at 37° C. After washing for 1 min, accumulated neutrophils were quantified. (A) Dose-dependent effect of SSL5 (0.3-10 μg/ml) on accumulation of neutrophils to PselFc. (B) Effect of 10 μg/ml SSL5 compared to the effect of 10 μg/ml anti-PSGL-1 KPL1, PL1 and PL2, and the W17/1 isotype control mAb directed against the C5aR. The data represent relative accumulation of neutrophils compared to control-treated cells and are mean values ±SEM of at least three independent experiments.

FIG. 7. Affinity of the SSL5 to PSGL-1 interaction. Direct binding of SSL5 to PSGL-1 at the protein level was determined through surface plasmon resonance (SPR) analysis. Different concentrations of SSL5 were presented to rPSGL/Ig coated on a SPR surface. SSL5 bound to rPSGL/Ig in a saturable and dose-dependent manner, and the apparent affinity constant (Kd) was calculated to be 0.82±0.54 μM

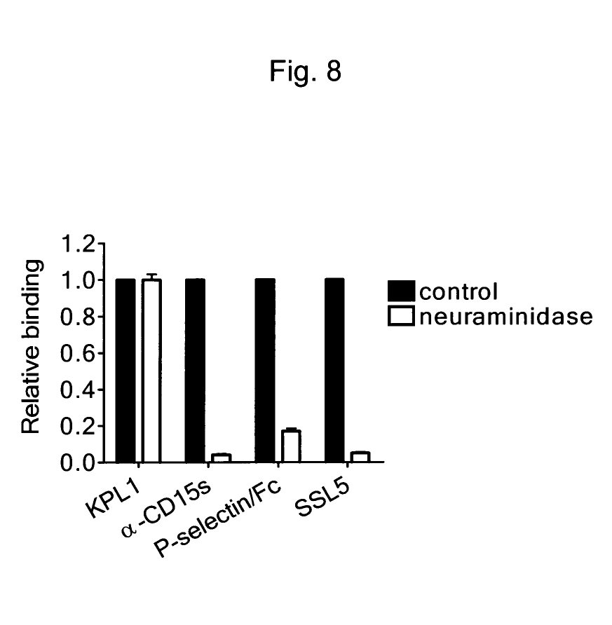

FIG. 8. sLex dependence of the SSL5-PSGL-interaction on the cell surface. PSGL-1-transfected CHO cells were also treated with neuraminidase to investigate the role of sialic acids. Upon treatment, PselFc and anti-CD15s binding were abolished, showing effective removal of sialic acids. SSL5 binding to treated CHO-PSGL-1 cells was also abrogated, suggesting sialic acid residues may be a critical determinant in recognition of PSGL-1 by SSL5. KPL1 binding remained equal compared to untreated cells as binding of this anti-PSGL-1 antibody is not sensitive to glycosylation.

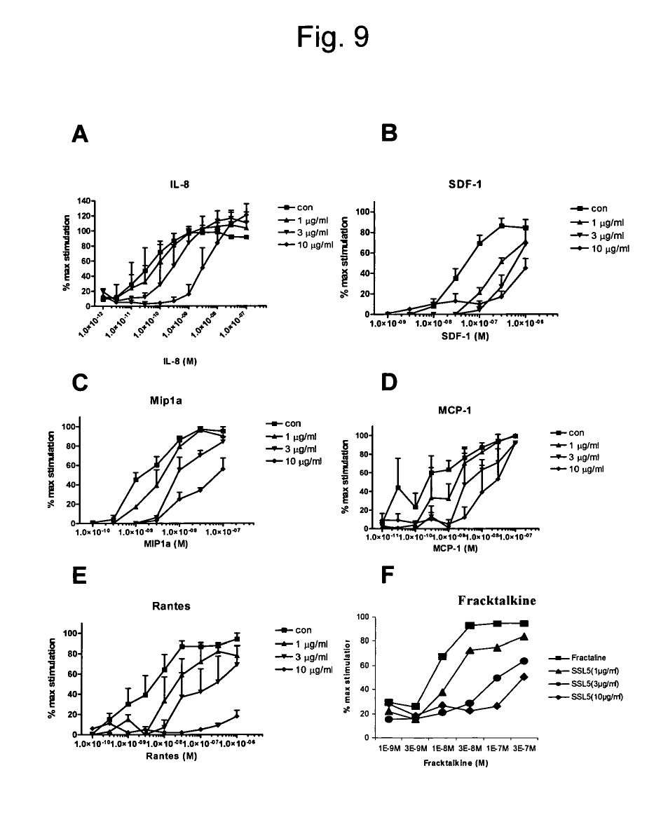

FIG. 9. SSL5 inhibits chemokine receptor activation. Cells (neutrophils or monocytes) were stimulated by the chemokines IL-8 (FIG. 9A), RANTES (FIG. 9B), MCP-1 (FIG. 9C), Mip-1alpha (FIG. 9D), and SDF-1 (FIG. 9E) and fractalkine (FIG. 9F) in various concentrations and the inhibitory effect of SSL5 at concentrations of 1, 3 and 10 μg/ml was measured by evaluating Calcium-mobilization.

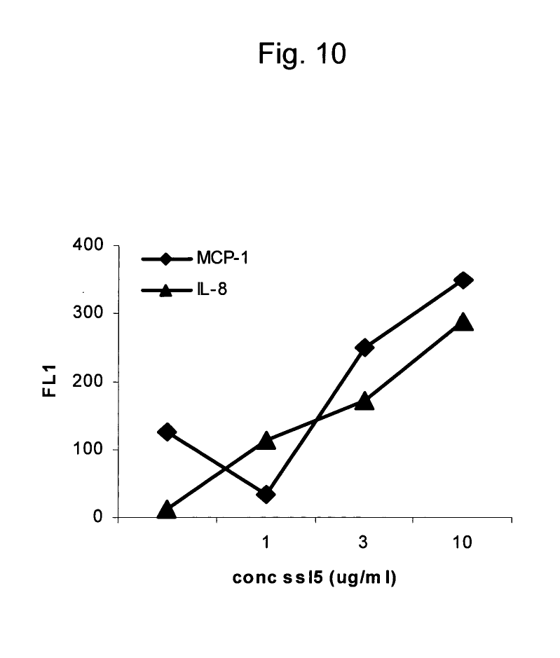

FIG. 10. SSL5 increases chemokine binding to cells To determine the effect of SSL5 on chemokine binding, the promonocytic cell line U937 that is devoid of chemokine receptors was used. This cell line does not bind chemokines, as was determined for IL-8 and MCP-1. Pretreatment of cells with SSL5 induced binding of these two chemokines in a dose-dependent manner.

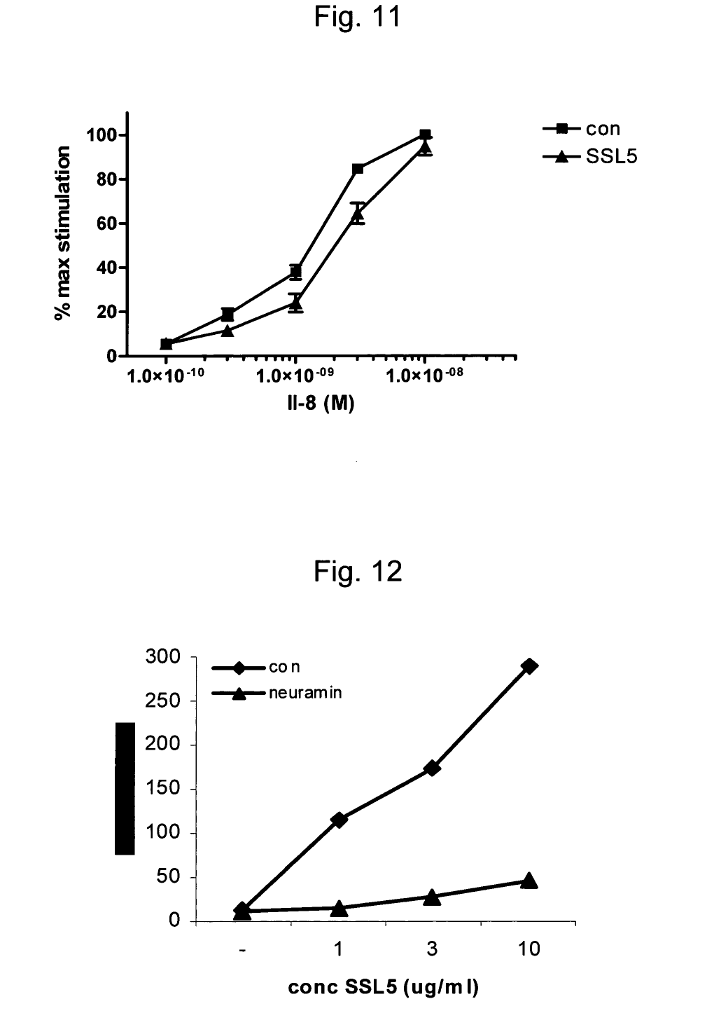

FIG. 11. SSL5 effect on chemokine-induced cell activation is sLex dependent. To determine whether SSL5 effect on cell activation by chemokines requires presence of sugar moieties at the cell surface, neutrophils were first treated with neuraminidase. This enzyme cleaves of sialic acids and disturbs the sLex moiety important for SSL5 binding to cells (FIG. 8). Treatment of cells with neuraminidase did not affect their stimulation by IL-8. However, their response could not be inhibited by SSL5 anymore, indicating an sLex-dependent effect of SSL5 on chemokine inhibition.

FIG. 12. SSL5 effect on chemokine binding is sLex dependent. Necessity of sLex for SSL5 effect on chemokine binding was also assessed on neuraminidase-treated U937-cells. While SSL5 increased binding of IL-8 to untreated cells, this effect was abolished upon treatment with neuraminidase (FIG. 12). Removal of sLex epitope thus abrogates the effect of SSL5 on chemokine binding.

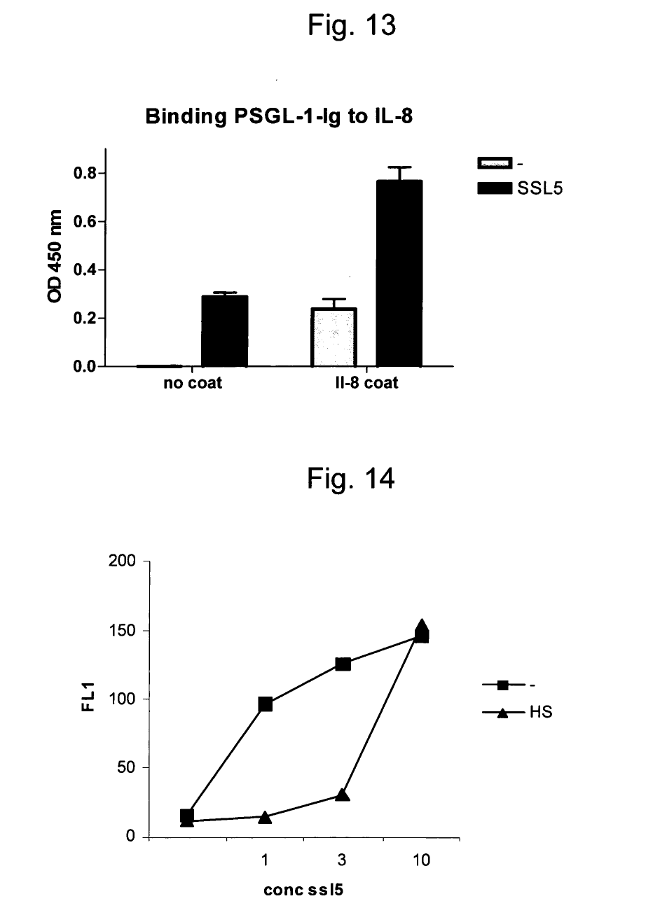

FIG. 13. SSL5 induces binding of IL-8 and PSGL-1 As SSL5 binding to cells as well as its effect on chemokine inhibition is sLex dependent, it was investigated whether SSL5 induces chemokine binding to a heavily glycosylated surface protein. For this purpose a surface was coated with IL-8 and binding of PSGL-1-Ig was determined in presence or absence of SSL5. FIG. 13 depicts that PSGL-1 does not bind to an uncoated or an IL-8-coated surfaces. However, SSL5 induced clear binding of PSGL-1 and IL-8.

FIG. 14. SSL5 and glycosaminoglycans compete for IL-8 binding site. Chemokines are generally presented on endothelial cells by glycosaminoglycans (GAG) such as heparin or heparan sulfate. To investigate whether SSL5 and GAGs compete for binding sites on the IL-8 molecules, IL-8 was first incubated with heparan sulfate and then added to SSL5-treated cells. FIG. 14 shows that increased binding of IL-8 by SSL5 was abolished when heparan sulfate-loaded IL-8 was used, indicating that SSL5 and heparan sulfate compete for binding of the chemokine.

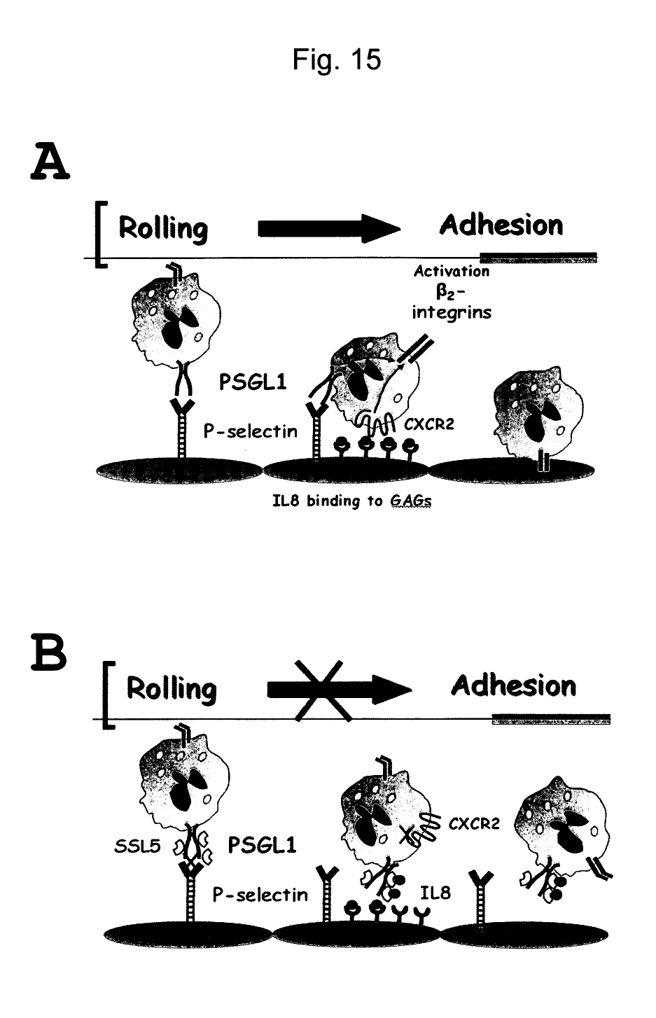

FIG. 15. Model of the mechanism of action of SSL5 in its anti-inflammatory action.

(A) Role for SSL5 in neutrophil extravasation. Initial neutrophil rolling on activated endothelial cells at inflamed sites is mediated by the interaction of PSGL-1 and P-selectin. Rolling allows for encounter of activating signals as IL-8 on the endothelial lining, which induce cell activation. Subsequent integrin upregulation enables firm adhesion of the cells and allows for their transmigration through the endothelial lining.

(B) SSL5 inhibits the two initial steps important in cell extravasation. Firstly, it inhibits neutrophil rolling by binding to PSGL-1. Secondly, SSL5 inhibits cell activation by binding to PSGL-1 and capturing the chemokines away from the chemokine receptors.

### EXAMPLES

### Example 1

### Binding of SSL5 to PGSL-1 on phagocytes

### Materials and Methods

### Antibodies

Phycoerythrin (PE)-conjugated monoclonal antibodies (mAb) directed against CD4, CD8, CD14, CD16, CD19, CD35, CD47, CD56, CD89, CD114, CDw119, CD132, CD142, and CD162, fluorescein isothyiocyanate (FITC)-labeled mAbs directed against CD11a, CD15, CD18, CD46, CD55, CD62L, CD66b, and CDw17, and allophycocyanin (APC)-labeled monoclonal antibodies against CD13, CD14, CD16, and CD45 were purchased from Becton Dickinson.

Monoclonal cyanine 5 (Cy5)-conjugated mAb against CD3 was purchased from Dako. CCR-1-PE, CCR-2-PE, CXCR-1-PE, CXCR-2-PE, TNF RI-FITC, and TNF RII-FITC were purchased from R&D Systems. CD10-APC was purchased from Caltag Laboratories (Burlingame, Calif.). CD63-PE was purchased from CLB.

The monoclonal anti-PSGL-1 antibodies PL1 (clones 3E2.25.5) and PL2 (clone 5D8.8.12) were obtained from Serotec, while KPL1 was purchased from Becton Dickinson.

A mAb against CD18 (IB4) was isolated from supernatant of mouse hybridoma obtained from American Type Culture Collection.

Monoconal antibody against C5aR (clone W17/1) was purchased from Serotec.

Polyclonal goat anti-mouse IgG-FITC was purchased from Southern Biotechnology, and goat anti-human IgG-FITC from Sigma Chemicals.

### Cloning, Expression and Purification of SSL5

For expression of recombinant SSL5, the SSL5 gene (ssl5) of S. aureus strain NTCT8325 except for the signal sequence was cloned into the expression vector pRSETB (Invitrogen) directly downstream of the enterokinase cleavage site. For this purpose, an overhang extension PCR was performed. First, the HIS tag and enterokinase cleavage site were amplified from the pRSETB vector using the XbaI recognition sequence and the N-terminal first 29 by sequence of ssl5 via the reverse primer (5′-GCTCTAGAAATAATTTTGTTTAACTTTAAGAAGGAG-3′, 5′-ACATTTTCATATTTTGCTTTATGTTCACTCTTGTCGTCATCGTCGTACAG-3′, XbaI recognition site underlined) introduced. Secondly, ssl5 was amplified by PCR (5′-AGTGAACATAAAGCAAAATATG-3′; 5′-CCGGAATTCTTATCTAATGTTGGCTTCTATTTTTTC-3′, EcoRI recognition site underlined) on DNA from S. aureus. Finally, a third PCR was performed to anneal the two PCR products together using the XbaI and EcoRI primer. All PCR products were amplified using PfuTurbo DNA polymerase (Stratagene).

After verification of the correct sequence, the pRSET/SSL5 expression vector was transformed in Rosetta-Gami (DE3) pLysS E. coli according to the manufacturer's protocol (Novagen). Expression of HIS-tagged SSL5 was induced with 1 mM isopropyl-β-D-thiogalactopyranoside (IPTG, Roche) for 3 hours. HIS-tagged SSL5 was isolated under denaturing conditions on a HiTrap chelating HP column according to the manufacturer's protocol (Amersham-Biosciences). The protein was renatured on the column by gradually exchanging denaturing buffer (8 M urea, 500 mM NaCl, 20 mM Na₂HPO₄, pH 5.3) for native buffer (500 mM NaCl, 20 mM Na₂HPO₄, pH 5.3). Bound protein was eluted using 50 mM EDTA. After dialysis, the HIS-tag was removed from SSL5 by cleavage with enterokinase according to manufacturer's instructions (Invitrogen).

### Cell Isolation

Leukocytes were isolated by means of the ficoll-histopaque gradient method. Venous blood was obtained from healthy volunteers using sodium heparin as anticoagulant (Greiner). Heparanised blood was diluted with an equal volume of phosphate buffered saline (PBS), and subsequently layered onto a gradient of Ficoll-Paque PLUS (Amersham Biotech) and Histopaque-1119 (Sigma-Aldrich). After centrifugation for 20 min at 400×g, plasma was aspired, and peripheral blood mononuclear cells (PBMC) were collected from the Ficoll layer and neutrophils from the Histopaque layer. After washing with RPMI 1640 containing 25 mM HEPES (N-2-hydroxyethyl-piperazine-N′-2-ethanesulfonic acid), L-glutamine (BioWhittaker) and 0.05% human serum albumin (HSA; CLB) (RPMI/HSA), the neutrophils were subjected to a hypotonic shock with water for 30 sec to lyse remaining erythrocytes, after which the neutrophils and PBMCs were washed.

### SSL5 Binding to Leukocyte Subpopulations

To determine binding of SSL5 to different leukocyte types, SSL5 was labeled with FITC. Therefore, 1 mg/ml SSL5 was incubated with 100 μg/ml FITC in 0.1 M sodium carbonate buffer (pH 9.6) for 1 h at 37° C. Using a HiTrap desalting column (Amersham Biosciences), labeled SSL5 was separated from unbound FITC. For binding of SSL5-FITC to leukocytes, neutrophils (5×10⁶ c/ml) and PBMCs (1×10⁷ c/ml) were incubated with increasing concentrations of SSL5-FITC in RPMI/HSA for 30 min on ice in presence of PE- or Cy5-conjugated, leukocyte-subset specific antibodies. Leukocyte-subset specific antibodies include anti-CD3, CD4, CD8, CD14, CD16, CD19, and CD56. After washing, fluorescence was measured on a flow cytometer (FACSCalibur, Becton Dickinson).

Competition for Receptor Binding of SSL5, anti-PSGL-1 and P-Selectin

To determine a putative receptor for SSL5, neutrophils (5×10⁶ c/ml) and PBMC (1×10⁷ c/ml) were incubated with 10 μg/ml SSL5 for 15 min on ice in presence of 10% heated human pooled serum. Subsequently, FITC-, PE- and APC-conjugated mAbs directed against a series of cell surface receptors were added for 30 min on ice. After washing, fluorescence was measured using flow cytometry. In another experiment, neutrophils (5×10⁶ c/ml) were incubated with increasing concentrations of SSL5 for 30 min on ice. After washing, the cells were incubated with 1 μg/ml anti-PSGL-1 mAbs or IB4 (isotype control) for 30 min on ice, washed and stained with 5 μg/ml goat anti-mouse IgG-FITC. For binding of KPL1 to subpopulations of leukocytes in competition with SSL5, cells were concurrently stained with PE- and Cy-conjugated, leukocyte-subset specific antibodies directed against CD3, CD4, CD8, CD14, CD16, CD19, and CD56. After washing, fluorescence was measured using flow cytometry. In a separate experiment, neutrophils were incubated with increasing concentrations of SSL5 or anti-PSGL-1 mAbs on ice for 30 min, washed and stained with 1 μg/ml PselFc chimera (PselFc; R&D Systems) for 30 min on ice. Bound PselFc was detected with 5 μg/ml goat anti-human IgG-FITC.

### Adhesion of Neutrophils to P-Selectin Under Static Conditions

To investigate adhesion of neutrophils to P-selectin, neutrophils were loaded with 4 μM calcein-AM (Molecular Probes) in Hank's buffered salt solution (HBSS, BioWhittaker) with 0.05% HSA (HBSS/HSA). A 96-wells plate (Greiner bio-one) was coated with 10 μg/ml PselFc for 1 h at 37° C. After washing with PBS, the plate was blocked with 4% BSA (Sigma) for 90 min at 37° C. The plate was then washed, and 3×10⁵ calcein-labeled neutrophils were added to duplicate wells and allowed to adhere for 15 min at room temperature. After washing, adherent cells were quantified using a platereader fluorometer (FlexStation, Molecular Devices).

### Adhesion of Neutrophils to P-Selectin Under Flow Conditions

To study the effect of SSL5 on the rolling of neutrophils, a flow chamber was used as previously described (Van Zanten et al. 1996. Blood 88:3862-3871). Briefly, the chamber is a modified form of transparent parallel-plate perfusion chamber in which a coverslip of 18 mm×18 mm is used as a rolling surface. Glass coverslips were coated with 10 μg/ml PselFc for 1 h at 37° C. After blocking with 1% HSA for 1 h, the slips were washed with PBS and inserted into the flow chamber. Individual perfusions were performed at a wall shear stress of 200/s by drawing cells through the perfusion chamber with a syringe pump (Harvard Apparatus, South Natick, Mass.). The perfusion chamber was mounted on a microscope stage (DM RXE; Leica, Weitzlar, Germany), which was equipped with a B/W CCD video camera (Sanyo, Osaka, Japan). To the camera a video recorder was connected to record individual perfusions. Recorded images were analysed by custom-made program using Optimas 6.1 software (Media Cybernetics systems, Silver Springs, Md.). The experiments were performed at 37° C.

Neutrophils were washed and diluted to 4×10⁶ c/ml in perfusion buffer (20 mM HEPES, 132 mM NaCl, 6 mM KCl, 1 mM MgSO₄, 1.2 mM KH₂PO₄, supplemented with 5 mM glucose, 1 mM CaCl₂, and 0.5% HSA). They were left at room temperature for 15 min before treatment with a concentration range of SSL5 (0.3-30 μg/ml) or 10 μg/ml anti-PSGL-1 for 15 min at 37° C.

The anti-C5aR antibody W17/1 was used as an isotype control antibody. Neutrophils were then diluted with perfusion buffer to 2×10⁶ c/ml and perfused for 5 min through the perfusion chamber containing the PselFc-coated coverslip. After washing with perfusion buffer for 1 min, the number of adherent neutrophils was quantified for at least 40 adjacent high power fields recorded along the perfusion chamber (total surface at least 1 mm²). Adherent PMN appeared as white-centred cells, and experiments in which more than 40 percent of the cells in control conditions flattened and did not appear as white-centred cells were discarded.

In addition, experiments in which adhesion of cells in control conditions was lower than 100 cells/mm² were also discarded.

### SSL5 Binding to Neuraminidase-Treated Cells

CHO-PSGL-1 cells (2×10⁶ cells/ml) were treated with 0.2 U/ml neuraminidase (from Vibrio cholerae, Sigma) at 37° C. for 45 minutes at pH 6.0. After washing, cells (0.5×10⁶ cells/ml) were stained for binding of SSL5-FITC, anti-sialyl Lewis x (CD15s) mAb, P-selectin/Fc. and anti-PSGL-1 mAb KPL1 for 30 minutes on ice.

### Surface Plasmon Resonance (SPR)-Analysis

SPR-analysis was performed employing a Biacore 2000 biosensor system (Biacore AB, Uppsala, Sweden). Streptavidin-sensor chips were loaded with biotinylated anti-Fcg F(ab′)₂ fragments (Jackson ImmunoResearch Europe, Sirham, United Kingdom) till a density of 2.5 kRU on two adjacent channels. The first of these channels (channel 1) was used as a control. Channel 2 was used to capture PSGL-1/Ig fusion protein (Pendu R et al. Blood. 2006; Pre-published Aug. 22, 2006; DOI 10.1182/blood-2006-03-010322). rPSGL/Ig was passed at a concentration of 0.3 mg/ml in 100 mM NaCl, 0.005% Tween-20, 2.5 mM CaCl₂, 25 mM Hepes (pH 7.4) at a flow rate of 5 μl/min at 25° C. to reach a density of 0.25 kRU. Subsequently, both channels were used for the perfusion of SSL5 at a flow rate of 5 μl/min in the same buffer until equilibrium was reached. Binding to the PSGL-1/Ig-coated channel was corrected for binding to the control channel.

Sensorgrams were analyzed using BIAevaluation-software provided by the manufacturer to determine responses at equilibrium (Req). Req was then plotted against protein concentration to calculate apparent affinity as described. (Romijn R A et al., J Biol. Chem. 2003; 278:15035-15039).

### Results

### SSL5 Binds to Different Leukocyte Populations

SSL5 was produced in Rosetta-Gami (DE3) pLysS E. coli and isolated with high purity. Fluorescein-labeled SSL5 (SSL5-FITC) and flow cytometry were used to determine its binding to different leukocyte populations (FIG. 1). Specific cell types were identified through scatter gates and cell type-specific lineage markers. Neutrophils (gated only on scatter), monocytes, and natural killer cells stained highly positive for SSL5-FITC, while binding to T lymphocytes was lower (FIG. 1A, B). Hardly any binding to B lymphocytes was observed.

### PSGL-1 is a Receptor for SSL5

A multi-screening assay for surface-expressed receptors of leukocytes was performed to identify a receptor for SSL5. For this purpose, a panel of thirty-one monoclonal antibodies was selected which recognise a variety of leukocyte receptors with distinctive functions including chemokine, cytokine and signalling receptors, and receptors involved in adhesion or phagocytosis. SSL5 was screened for its ability to block binding of these antibodies to neutrophils, monocytes, and lymphocytes in presence of serum. SSL5 competitively blocked binding of anti-PSGL-1 (CD162) on neutrophils and monocytes but not on lymphocytes (FIG. 2). Binding of antibodies directed against the other thirty cell surface receptors was not significantly affected by SSL5. Thus, PSGL-1 was identified as a putative receptor for SSL5.

SSL5 Competes with Antibodies Directed Against PSGL-1

To address the interaction of SSL5 and PSGL-1 more directly, SSL5 was screened for its ability to block binding of several anti-PSGL-1 mAbs to neutrophils. SSL5 competitively inhibited binding of all three anti-PSGL-1 mAbs tested in a dose-dependent manner (FIG. 3A-B). Binding of PL1 and KPL1 (two blocking antibodies) was strongly inhibited by SSL5. Binding of PL2 (a non-blocking antibody) was also inhibited but to a lower extent; ten-fold more SSL5 was needed to achieve the same inhibition compared to PL1 or KPL1. Staining of neutrophils with isotype control mAb IB4 directed against beta-2 integrins was not affected by SSL5. Binding to PSGL-1 was specific for SSL5, as five other SSLs tested showed no competition with anti-PSGL-1 mAbs in this assay (data not shown).

In addition to neutrophils, competition of SSL5 and KPL1 was also examined on other leukocyte subpopulations. SSL5 also inhibited binding of KPL1 to monocytes and natural killer cells and hardly on T or B lymphocytes. Thus, competition was observed on those cells which bind SSL5 well (FIG. 3C). Good competition of SSL5 and KPL1 on natural killer cells and some competition on T lymphocytes (FIG. 3C) compared to lack of competition of SSL5 and KPL1 to the general lymphocyte population in the multiscreening assay (FIG. 1B) can be accounted for by absence of serum in this experiment.

### SSL5 Inhibits Binding of P-Selectin to Neutrophils

To investigate whether SSL5 interferes with the interaction of PSGL-1 and its ligand P-selectin, flow cytometry was used to measure binding of P-selectin/Fc chimera (PselFc) to neutrophils. SSL5 was incubated in a serial dilution with neutrophils with a fixed concentration of PselFc. SSL5 clearly inhibited binding of PselFc (FIG. 4A). This effect was dose-dependent and comparable to the anti-PSGL-1 mAb KPL1 (FIG. 4B). PL1, but not PL2, slightly blocked binding of Psel-Fc to neutrophils.

### SSL5 Blocks Adhesion of Neutrophils Under Static Conditions

Adhesion assays were performed to determine if SSL5 also inhibits the cell adhesion function of PSGL-1. Under static conditions, binding of neutrophils to a PselFc-coated surface was analysed in the presence or absence of SSL5 or anti-PSGL-1 mAbs. In agreement with the results obtained from flow cytometry, SSL5 strongly inhibited binding of neutrophils to PselFc. Maximal inhibition of 80% was achieved at a concentration of 3 μg/ml SSL5 (FIG. 5). KPL1 also effectively blocked neutrophil adhesion to PselFc, while PL1 inhibition was weaker and no effect was observed for PL2.

### SSL5 Inhibits Adhesion of Neutrophils Under Shear Conditions

The effect of SSL5 on rolling of neutrophils on P-selectin was examined under physiological shear stress by means of a parallel-plate perfusion chamber. In this chamber, neutrophils were perfused over glass cover slips coated with PselFc at a shear stress of 200/s in presence or absence of SSL5 or anti-PSGL-1 mAbs. After 5 minutes, the number of accumulated neutrophils per mm² was determined. In absence of SSL5, neutrophils adhered efficiently to PselFc. Treatment of neutrophils with SSL5 strongly inhibited their binding under flow conditions in a dose-dependent manner (FIG. 6A). The inhibitory effect of SSL5 on neutrophil rolling was comparable to that of PL1 and PL2, while KPL1 completely abolished adhesion (FIG. 6B). Treatment of neutrophils with isotype control mAb W17/1 directed against the C5aR had no effect on rolling of neutrophils on the PselFc surface.

### Interaction Between SSL5 to PSGL-1

To get a better idea of the exact interaction between SSL5 and PSGL-1, direct binding of SSL5 to PSGL-1 at the protein level was determined through surface plasmon resonance (SPR) analysis on a Biacore system (FIG. 7).

A recombinant human PSGL-1-Fc construct was immobilized on a SPR surface. Subsequently different concentrations of SSL5 were presented to this coated surface. SSL5 bound to rPSGL/Ig in a saturable and dose-dependent manner, and the apparent affinity constant (Kd) was calculated to be 0.82±0.54 μM. This affinity constant is in the same range as the reported PSGL-1 to P-selectin interaction, thereby making it very likely that SSL-5 can disturb the interaction between PSGL-1 and P-selectin.

### The Role of Sialic Acid Residues

PSGL-1-transfected CHO cells were also treated with neuraminidase to investigate the role of sialic acid residues (sialic acid is a crucial part of the sLex epitope on PSGL-1 and other cell surface proteins). Indeed, as demonstrated in FIG. 8, upon treatment with neuraminidase P-selectin/Fc and anti-CD15s binding were abolished, showing effective removal of sialic acids. Also upon treatment, SSL5 binding to treated CHO-PSGL-1 cells was also abrogated, suggesting sialic acid residues may be a critical determinant in recognition of PSGL-1 by SSL5. KPL1 binding remained equal compared to untreated cells as binding of this anti-PSGL-1 antibody is not sensitive to glycosylation.

So the SSL5 binds to PSGL-1 on the cell surface of phagocytes, specifically to sLex residues, and thereby inhibits the interaction with P-selectin, causing the inhibition of neutrophil rolling.

### Example 2

### Inhibition of Chemokines by SSL5

### Materials and Methods

### Calcium Mobilization

Calcium mobilization with isolated human neutrophils was performed as follows. Neutrophils or PBMC's were loaded with 2 μM Fluo-3-AM in RPMI containing 0.05% human serum albumin (HSA) for 20 minutes under agitation at room temperature, washed with buffer and suspended to 10⁶ cells/ml in RPMI/HSA. Fluo-3-loaded cells were incubated with or without 1 to 10 μg/ml of SSL5. Then, basal calcium levels were measured in the FACSCalibur flow cytometer (Becton Dickinson), after which a concentration gradient of agonist (10⁻¹² to 10⁻⁷ M of IL8, RANTES, MCP-1, Mip1-alpha, SDF-1, or fractalkine) was added.

Samples were analysed after gating the neutrophil population, thereby excluding cell debris and background noises. The calcium flux response of the cells for each concentration of agonist was expressed as mean fluorescence. In some experiments U937 cells transfected with the CXCR2 were used using the same method as described for the neutrophils.

### Chemokine Binding to Cells

U937 cells (5×10⁶ cells/ml) were incubated with an increasing concentration of SSL5 for 15 min on ice. Subsequently, biotin-labeled IL-8 or MCP-1 was added for 30 min according to manufacturer's protocol (R&D Systems). Bound IL-8 or MCP-1 was detected with streptavidin-FITC by means of flow cytometry.

In another experiment, cells were first treated with 0.2 U/ml neuraminidase (from Vibrio cholerae, Sigma) at 37° C. for 45 minutes at pH 7.4. Where indicated, biotin-labeled IL-8 was first preincubated with 1 mg/ml heparan sulfate.

### Elisa

Wells (Microsorp U96 Elisa plate, Nunc) were coated with PBS or IL-8 (Peprotech) for 2 h at 37° C., and blocked for 15 min at room temperature with blocking reagent (Roche). 1 μg/ml PSGL-1/Ig was added in the presence or absence of 10 μg/ml SSL5 for 1 h at 37° C. in Tris-buffer (50 mM Tris, 1 mM CaCl₂, ZnCl, 0.05% Tween, pH 7.4). Detection of bound PSGL-1-Fc was performed with subsequent incubations with Biotin-SP-AffiniPure IgG Frag Goat anti-human F(ab′)₂ (Sanbio) and streptavidin-HRPO (Dako) for 30 min at 37° C. in Tris buffer.

### Results

### SSL5 Inhibits Chemokine Receptor Signalling

The chemokine receptors all belong to the family of G-protein coupled receptors (GPCR). Ligands for the specific receptors that were used in this example are as follows:

IL-8 for the receptors CXCR1 and CXCR2;

RANTES for CCR1, CCR3 and CCR5;

MCP-1 for the receptor CCR2;

Mip-1alpha for CCR1 and CCR5;

SDF-1 for CXCR4; and

fractalkine for the receptor CXC3CR1.

These ligands are important chemokines produced at a site of inflammation and able to attract leukocytes to the site of inflammation. Furthermore, they play an important role in the activation of rolling neutrophils resulting in the activation of neutrophil β2-integrins and subsequent firm adhesion of these integrins to the activated endothelial cell layer. This is an essential step in the extravasation process of neutrophils towards the site of inflammation.

Calcium mobilization is one of the hallmarks of GPCR activation with its ligands. It is shown in FIG. 9 that SSL-5 next to its effect on PSGL-1 as described in Example 1, also inhibits chemokine receptor activation.

Cells (neutrophils or PBMC), stimulated by the chemokines IL-8 (FIG. 9A), RANTES (FIG. 9B), MCP-1 (FIG. 9C), Mip-1alpha (FIG. 9D), SDF-1 (FIG. 9E) and fractalkine (FIG. 9F) in various concentrations and the inhibitory effect of SSL5 at concentrations of 1, 3 and 10 μg/ml was measured by evaluating calcium mobilization.

Cell activation by all these chemokines was inhibited by SSL5. As a control, cells were stimulated by non-chemokine chemoattractants as PAF, LTB4, fMLP and C5a, that stimulate different GPCR's. These are not inhibited by SSL5. This inhibition is thus specific for the chemokines of the CC, CXC and CX3C family.

The mechanism by which the inhibition of this great variety of chemokines is achieved is related to the SSL5-PSGL-1 interaction as described in Example 1. This will become apparent in the following series of experiments. First, chemokines are bound to the surface of phagocytes in the presence of SSL5. As shown in FIG. 10 SSL5 increases chemokine binding to the promonocytic cell line U937. This cell does not have any chemokine receptors, so direct interaction between chemokines and the cell is not possible. This is shown for IL-8 and MCP-1. Pretreatment of cells with SSL5 induced binding of these two chemokines in a dose-dependent manner.

Furthermore, this interaction of chemokines with the cells in the presence of SSL5 is sLex dependent as is demonstrated in FIG. 11. Neutrophils were first treated with neuraminidase. This enzyme cleaves of sialic acids and disturbs the sLex moiety important for SSL5 binding to cells (as was already shown in FIG. 8). Treatment of cells with neuraminidase did not affect their stimulation by IL-8. This indicates that the normal chemokine receptor-chemokine interaction was not disturbed. However, this response could no longer be inhibited by SSL5. This clearly demonstrates, an sLex-dependent effect of SSL5 on chemokine inhibition.

Next to this activation, also the SSL5 mediated binding of chemokines is sLex dependent. While SSL5 increased binding of IL-8 to untreated cells, this effect was abolished upon treatment with neuraminidase (FIG. 12). Removal of an sLex epitope thus abrogates the effect of SSL5 on chemokine binding.

From the results it is concluded that a three-molecular complex is formed on the surface of the cell. SSL5 binds chemokines, but only when associated with PSGL-1, or other heavily sLex-loaded surface proteins. To test this hypothesis, a surface was coated with IL-8 and binding of PSGL-1-Ig was determined in the presence or absence of SSL5. FIG. 13 depicts that PSGL-1 does not bind to an uncoated or an IL-8-coated surface. However, SSL5 induced clear binding of PSGL-1 and IL-8.

If indeed it is the case that SSL5, when bound to sLex-loaded surface molecules of phagocytes, binds chemokines, and thereby inhibits the activation of chemokine receptors, SSL5 should also disturb the original interaction of chemokines with the GAGs on endothelial cells. Chemokines are generally presented on endothelial cells by GAGs such as heparin or heparin sulfate. If this interaction would not be disturbed, the phagocyte would still be attached to the endothelium. To test whether SSL5 and GAGs compete for binding sites on the IL-8 molecules an assay was developed wherein IL-8 was first incubated with heparin sulfate and then added to SSL5-treated cells. FIG. 14 shows that increased binding of IL-8 by SSL5 was abolished when heparin sulfate-loaded IL-8 was used, indicating that SSL5 and heparin sulfate compete for binding of the chemokine.

### Model for the Mechanism of Action of SSL5 in its Anti-Inflammatory Action

All the experiments described so far, lead to the following model for the mechanism of action of SSL5 in its anti-inflammatory action and thus the role for SSL5 in the inhibition of phagocyte extravasation. As shown in FIG. 15A initial neutrophil rolling on activated endothelial cells at inflamed sites is mediated by the interaction of PSGL-1 and P-selectin. Rolling allows for encounter of activating signals as IL-8 on the endothelial lining (bound to GAG's), which induce cell activation. Subsequent integrin upregulation enables firm adhesion of the cells and allows for their transmigration through the endothelial lining.

SSL5 inhibits the two initial steps important in cell extravasation (FIG. 15B). Firstly, it inhibits neutrophil rolling by binding to PSGL-1. Secondly, SSL5 inhibits cell activation by binding to PSGL-1 and capturing the chemokines away from the chemokine receptors.

Thus, SSL5 inhibits the first two crucial steps in phagocyte extravasation. It inhibits the interaction of P-selectin with PSGL-1 and any other heavily and properly glycosylated phagocyte surface molecule. In doing so, it also gains affinity for GAG-bound chemokines of at least the CC, CXC and the CX3C class and displaces these from the GAGs, keeping them bound to the PSGL-1-SSL5 complex. Now also the second step in cell migration and cell activation is abrogated. The combination of these two effects make SSL5 a very strong, highly potent, and completely unique anti-inflammatory molecule.

1. Staphylococcal superantigen-like protein 5 (SSL5) or homologues or derivatives thereof for use in medicine.

2. Staphylococcal superantigen-like protein 5 (SSL5) as claimed in claim 1 having the amino acid sequence according to SEQ ID NO:1.

3. Staphylococcal superantigen-like protein 5 (SSL5) or homologues or derivatives thereof as claimed in claim 1 for use in the treatment of indications involving an excessive recruitment of leukocytes.

4. Staphylococcal superantigen-like protein 5 (SSL5) or homologues or derivatives thereof as claimed in claim 3, wherein the indications are selected from stroke, perfusion/ischemia, transplant rejection, rheumatoid arthritis.

5. Staphylococcal superantigen-like protein 5 (SSL5) or homologues or derivatives thereof as claimed in claim 1 for use as a PSGL-I inhibitor.

6. Staphylococcal superantigen-like protein 5 (SSL5) or homologues or derivatives thereof as claimed in claim 1 for use as an inhibitor of chemokine stimulation of phagocytes.

7. Staphylococcal superantigen-like protein 5 (SSL5) or homologues or derivatives thereof as claimed in claim 1 for use as an inhibitor of the interaction of P-selectin with PSGL-I and an agent for capturing GAG-bound chemokines.

8. Staphylococcal superantigen-like protein 5 (SSL5) or homologues or derivatives thereof as claimed in claim 1, wherein the homologue is an allelic variant of the S. aureus SSL5.

9. Staphylococcal superantigen-like protein 5 (SSL5) or homologues or derivatives thereof as claimed in claim 8, wherein the homologue is an SSL according to SEQ ID NO:2.

10. Staphylococcal superantigen-like protein 5 (SSL5) or homologues or derivatives thereof as claimed in claim 1, wherein the homologue is an homologue from another bacterial strain.

11. Staphylococcal superantigen-like protein 5 (SSL5) or homologues or derivatives thereof as claimed in claim 1, wherein the derivative is a polypeptide or peptide comprising a fragment of consecutive amino acids of the sequence shown in SEQ ID NO:1 or SEQ ID NO: 2, which fragment is an inhibitor of the interaction of P-selectin with PSGL-I and/or an agent for capturing GAG-bound chemokines.

12. Pharmaceutical composition comprising a suitable excipient and a therapeutically active amount of Staphylococcal superantigen-like protein 5 (SSL5) or a homologue or derivative thereof.

13. Pharmaceutical composition as claimed in claim 12, wherein the Staphylococcal superantigen-like protein 5 (SSL5) has the amino acid sequence according to SEQ ID NO:1.

14. Pharmaceutical composition as claimed in claim 12 for the treatment of indications involving an excessive recruitment of leukocytes.

15. Pharmaceutical composition as claimed in claim 14, wherein the indications are selected from stroke, perfusion/ischemia, transplant rejection, rheumatoid arthritis.

16. Pharmaceutical composition as claimed in claim 12 for use as a PSGL-I inhibitor.

17. Pharmaceutical composition as claimed in claim 12 for use as an inhibitor of chemokine stimulation of phagocytes.

18. Pharmaceutical composition as claimed in claim 12 for use as an inhibitor of the interaction of P-selectin with PSGL-I and an agent for capturing GAG-bound chemokines.

19. Pharmaceutical composition as claimed in claim 12, wherein the homologue is an allelic variant of the S. aureus SSL5.

20. Staphylococcal superantigen-like protein 5 (SSL5) or homologues or derivatives thereof as claimed in claim 19, wherein the homologue is an SSL according to SEQ ID NO: 2.

21. Staphylococcal superantigen-like protein 5 (SSL5) or homologues or derivatives thereof as claimed in claim 12, wherein the homologue is an homologue from another bacterial strain.

22. Staphylococcal superantigen-like protein 5 (SSL5) or homologues or derivatives thereof as claimed in claim 12, wherein the derivative is a polypeptide or peptide comprising a fragment of consecutive amino acids of the sequence shown in SEQ ID NO:1 or SEQ ID NO: 2, which fragment is an inhibitor of the interaction of P-selectin with PSGL-I and/or an agent for capturing GAG-bound chemokines.

23. Use of Staphylococcal superantigen-like protein 5 (SSL5) or homologues or derivatives thereof for the preparation of a medicament for the treatment of indications involving an excessive recruitment of leukocytes.

24. Use as claimed in claim 23, wherein the indications are selected from stroke, perfusion/ischemia, transplant rejection, rheumatoid arthritis.


SEQUENCE LISTING (6 sequences)

SEQ ID NO: 1 - protein, 204 aa, Staphylococcus aureus
SEHKAKYENVTKDIFDLRDYYSGASKELKNVTGYRYSKGGKHYLIFDKNRKFTRVQIFGKDIERFKARKNPGLDIFVVKEAENRNGTVFSYGGVTKKNQDAYYDYINAPRFQIKRDEGDGIATYGRVHYIYKEEISLKELDFKLRQYLIQNFDLYKKFPKDSKIKVIMKDGGYYTFELNKKLQTNRMSDVIDGRNIEKIEANIR

SEQ ID NO: 2 - protein, 204 aa, Artificial
  example of homologue of SSL5 (SEQ ID No 1) of S.aureus
SEHKAKYENVTKDIFDLRDYYSGASKELKNVTGYRYSKGGKHYLIFDKHQKFTRIQIFGKDIERLKTRKNPGLDIFVVKEAENRNGTVFSYGGVTKKNQGAYYDYLNAPKFVIKKEVDAGVYTHVKRHYIYKEEVSLKELDFKLRQYLIQNFDLYKKFPKDSKIKVIMKDGGYYTFELNKKLQPHRMSDVIDGRNIEKMEANIR

SEQ ID NO: 3 - DNA, 36 bp, Artificial
  SSL5 reverse primer with XbaI site
gctctagaaataattttgtttaactttaagaaggag

SEQ ID NO: 4 - DNA, 50 bp, Artificial
  reverse primer SSL5 II
acattttcatattttgctttatgttcactcttgtcgtcatcgtcgtacag

SEQ ID NO: 5 - DNA, 22 bp, Artificial
  amplification primer SSL5 I
agtgaacataaagcaaaatatg

SEQ ID NO: 6 - DNA, 36 bp, Artificial
  amplification primer SSL5 II
ccggaattcttatctaatgttggcttctattttttc